(19) United States
(12) Patent Application Publication
(10) Pub. No.: US 2005/0196469 A1
(43) Pub. Date: Sep. 8, 2005

(54) Micronutrient supplement combination for acne treatment and prevention

(76) Inventor: Susan Thys-Jacobs, Larchmont, NY (US)

Correspondence Address: GOTTLIEB RACKMAN & REISMAN PC, 270 MADISON AVENUE, 8TH FLOOR, NEW YORK, NY, 100160601, US

(21) Appl. No.: 10/794,729

(22) Filed: Mar. 4, 2004

(51) Int. Cl. A61K 31/59; A61K 31/525; A61K 33/10; A61K 31/19

(52) U.S. Cl. 424/687; 514/168; 514/251; 514/574

(57) ABSTRACT

A micronutrient supplement comprising effective amounts of calcium, vitamin D, and folate treats and prevents acne.

### FIELD OF THE INVENTION

This invention relates to a micronutrient supplement in the treatment of acne vulgaris and inflammation. In particular, this invention relates to a multi-vitamin and mineral supplement for improving skin and hair health.

### BACKGROUND OF THE INVENTION

Acne is a common problem affecting adolescents and young adults. It is the most common skin disease treated by physicians. Approximately 17-45 million people in the US are afflicted with acne and although associated with the very young, it can continue into well into adult life. It is often self-limited but can become chronic and result in emotional distress and permanent scarring. The reported prevalence of acne varies from 35 to 90% of adolescents at some stage. The prevalence of acne varies between sexes and age groups, appearing earlier in females than in males, possibly reflecting the earlier onset of puberty in females with a greater severity later in males as androgens levels rise. Acne occurs in up to 50% of adolescent girls and 85% of boys. Some studies have shown a seasonal variation in acne with an exacerbation during the colder months and an improvement during the warmer months.

Acne is a disorder of the pilosebaceous follicles, sebaceous glands and rudimentary or vellus hair involving the face, chest and back. The precise pathogenesis is not known. Androgens have been linked to stimulation of sebum production. Androgen stimulates both sebaceous gland cell division and intracellular lipid synthesis. Estradiol, on the other hand, appears to have a potent inhibitory effect on sebum production and little or no effect on cell division. Multiple complex factors have a role involving abnormal keratinization, hormonal function, bacterial growth and immune hypersensitivity. These factors include increased sebum production, comedo formation, colonization of the follicle by Propionibactrium and the host's inflammatory response.

The primary acne lesion is the blackhead, an impaction and distension of the follicle with desquamated keratinocytes and sebum. Hormonal changes at puberty, specifically androgens, can stimulate the production of sebum and trigger acne lesions. Propionibacterium acnes is an anaerobic, gram positive bacterium that colonizes the pilosebaceous follicles following the increases in sebum production with the accompanying hormonal changes, attracts polymorphonuclear neutrophils which release hydrolytic enzymes and contributes to the causation of acne. Propionibacterium acnes also contributes to the inflammatory process by hydrolyzing triglycerides to free fatty acids and by increasing follicular obstruction. Genetics appears to have some role in the severity of acne.

Acne can develop into disfiguring scars or cysts as inflammatory lesions resolve. Even small inflammatory lesions may produce scars. Acne treatment is a long term process that when abandoned, may result in the return of acne vulgneiform eruptions. To date, there has been no cure. However, there are numerous topical and oral treatments that are effective in the management of acne. Current acne treatment entails topical and systemic preparations. Topical therapies include benzoyl peroxide which has comedolytic and antibacterial effects, topical antibacterials such as erythromycin or clindamycin, azelaic acid, tazaroc, and topical retinoids. Acne that is resistant to topical treatment requires oral antibiotics or isotretinoin. Indications for isotretinoin include severe scarring, acne that is resistant to oral antibiotics and acne present for many years that quickly relapses when an oral antibiotic therapy is discontinued. Of note, oral isotretinoin is a potent teratogen. Current standards of acne therapy include the topical desquarnative drugs and antibacterial agents. In women, anti-androgen therapy for acne management has been relatively successful and this may involve oral contraceptives, spironolactone, and cyproterone.

Very little is known concerning diet and acne. In fact, diet has never been shown to have much effect on acneiform eruptions although inadequate folate intake (less than 400 micrograms daily) has been cited in one reference to be associated with acne formation, (although the recommended quantity would not usefully treat acne) while excess of the other B vitamins have erupted into acne. One citation from Sheretz describes a severe case of acneiform eruption that was temporally associated with high dose B6 and B12 supplementation that resolved and promptly improved with discontinuation of the vitamin complex. (Sheretz E F, “Acneiform eruption due to vitamins B6 and B12”, CUTIS 1991; 48(2):119-20). Some vitamin deficiencies have been associated with dermatitis and skin inflammation. An abnormal ratio of folate to B6 to B12 may have resulted in this acne eruption and inflammation or merely the absence of adequate folate ingestion. Acne formation, sore tongue, cracking at the corners of the mouth have all been associated with one or many deficiencies of the B vitamins such as riboflavin, folate, and pyridoxine.

The skin appears to be a very important source of vitamin D synthesis and may even have a role in the catabolism of the B vitamin, folate. (B Cohn. “Sunlight, skin color, and folic acid.” J Am Acad Dermatol. 2002; 46:31-8.) Skin color may also pose a greater or lesser risk of either vitamin D deficiency or folate photolysis based on pigment. Darker skin has increased melanin with greater protection against ultraviolet radiation and skin cancer risk, but less of an ability to produce previtamin D from a give amount of ultraviolet radiation B (UVB). Lighter skin has the ability to synthesize more vitamin D from UVB but has a greater risk for skin cancer because of less melanin pigment. Branda and Eaton in 1978 proposed that significant ultraviolet radiation may result in chemical breakdown and photolysis of folate. (Branda R F. “Skin Color and nutrient photolysis: an evolutionary hypothesis. Science 1978; 201:625-6). Darker skin appears to protect against ultraviolet radiation induced folate photolysis.

Vitamin and mineral preparations are commonly administered to treat specific medical conditions or as general nutritional supplements. Micronutrients are elements or compounds which are present in foods in small or trace amounts and include vitamins, minerals, or other elements. The macronutrients comprise carbohydrates, fats, and proteins which supply nutrients and calories. The primary source of all nutrients is of course food. However, the majority of people do not meet the Recommended Dietary Allowance—RDA of the foods containing these essential compounds and elements. Thus vitamin and mineral supplementation has become a recognized method of meeting accepted medical and health standards.

Vitamin D is a fat soluble vitamin that is rarely found naturally in food. It is not a true vitamin, but is a steroid prohormone that is produced in the skin by ultraviolet sunlight and converted by a series of hydroxylations to a biologically active steroid hormone metabolite (M. Peacock. “Effect of calcium and vitamin D insufficiency on the skeleton. Osteoporosis Int. Suppl 8: S45-S51. (1998); Reinhold Vieth. “Vitamin D supplementation, 25-hydroxyvitamin D concentrations and safety.” Am J Clin Nutrition, vol 69, pp. 842-56. (1999). (herein Peacock M; Vieth R). Vitamin D is a major regulator of calcium homeostasis and bone metabolism. However, only recently has it been hypothesized that ingestion of adequate concentrations of vitamin D that maintain vitamin D in the sufficiency range can result in superior bone health and vitality. There have been many reported benefits of vitamin D such as in the prevention of osteomalacia, osteoporosis, breast and colon cancer, osteoarthritis progression and hypertension. Recent evidence suggests that vitamin D may have anti-inflammatory and immunosuppressive effects. In one review, (PC van de Kerkhof. “Biological activity of vitamin D analogues in the skin, with special reference to antipsoriatic mechanisms” Brit J Derm. Vol 132. pp 675-82. (1995)), active vitamin D was noted to modulate epidermal growth, keratinization and inflammation and proved effective in the treatment of the skin disease, psoriasis. Calcitriol, an active metabolite of vitamin D was noted to decrease keratinocyte proliferation, normalize keratinocyte differentiation and decrease immune activation in plaques (I Lu et al. “Modulation of epidermal differentiation, tissue inflammation, and T-lymphocyte infiltration in psoriatic plaques by topical calcitriol”. J Cutaneous Pathology. Vol 23, pp 419-30. (1996)). Active vitamin D appeared to suppress immune and keratinocyte activation. Another study by Matsuyama and colleagues (W Matsuyama.” Idiopathic Hypoparathyroidism with fungal seminal inflammation. Internal Medicine.” 36: 113-7; 1997) suggested that active vitamin D may possess immunological effects. Abnormalities of calcium regulation with low calcium and parathyroid hormone concentrations as described in a patient with idiopathic hypoparathyroidism resulted in fungal infections which were successfully treated with the anti-fungal treatment fluconazole only when the patient was administered active vitamin D therapy. Muller et al. reported that proliferation of T-cells and their release of cytokines such as IL-2 and interferon gamma were also suppressed by active vitamin D. (K Muller et al. “1,25dihydroxyvitamin D3 as a natural regulator of human immune functions”. Journal Investigative Dermatology. Vol 1, pp. 68-71 (1996)). Vitamin D insufficiency defined as a serum 25 hydroxyvitamin D concentration below 40 ng/ml can result in reduced calcium supplies, accelerated bone turnover and suboptimal bone mass and mineralization (Peacock M.). Levels of 25 hydroxyvitamin D above 9-10 ng/ml were for many years believed to be sufficient and optimal for calcium homeostasis and bone health with the RDA (recommended dietary allowance) at 400 IU corresponding to a safe and adequate intake. Because vitamin D is lipid soluble and potentially toxic, oral intakes of vitamin D greater than 1000 IU have not been advised. One report cites that ingestion of parent vitamin D, cholecalciferol, is safe at daily doses of 2000 IU up to 10,000 IU daily with toxicity occurring at doses of 40,000 IU daily (Vieth R). Attaining a 25 hydroxyvitamin D concentration above 40 mg/ml may involve an intake of more than 2000 IU daily.

Calcium has been shown to be particularly effective in improving health. It is an essential mineral nutrient that is necessary on a daily basis for numerous key physiologic functions in the body, including nerve, muscle, skin, endocrine functions acting as an important second messenger. Deficiencies of calcium can have broad ranging adverse effects on many tissues and may manifest clinically as irritability, muscle spasm, myalgias, fatigue, anxiety and depression. Adequate dietary calcium intake has been shown to reduce bone resorption, osteoporosis and fracture risk. Chronic low dietary calcium intake results in low bone mass in many animal investigations, while calcium supplementation leads to increased bone mass (Peacock M). Calcium is required in supporting the bone formation phase of bone remodeling and is essential in bone growth, in optimizing peak bone mass and in the mineralization of the skeleton (Bess Dawson Hughes. “Effect of calcium and vitamin D supplementation on Bone Mineral Density in men and women 65 years of age or older” New England Journal of Medicine. Vol 337. pp. 670-6. (1997)). Inadequate and low calcium intake can influence optimum fracture healing and probably healing in general. (Kubo T. Et al. “Osteoporosis influences the late period of fracture healing in a rat model prepared by ovariectomy and low calcium diet.” Journal Steroid Biochemistry & Molecular Biology. 1999 Vol 68. pp. 197-202). The current DRI (Dietary Reference Intake) for adults younger than 50 years is 1000 mg of elemental calcium daily; and for adults older than 50 the DRI is 1500 mg daily of calcium.

The B Vitamins are important in cell division and cell growth, red cell formation, DNA/RNA synthesis, and energy production. Deficiency of folate has been associated with spina bifida, fatigue, acne, cheilitis, glossitis and mouth ulcers. Deficiency of vitamin B6 has been associated with depression, dermatitis and glossitis. Deficiency of B12 has been linked to psychiatric, hematological and neurological disturbances. The B vitamins also appear important via their effects on homocysteine levels, in coronary artery disease and risk, in the pathogenesis of vascular damage, and in inflammation. Homocysteine is a homolog of cysteine and is produced by the demetbylation of methionine, and is an intermediate in the biosynthesis of cysteine from methionine via cystathionine. Homocysteine is not inherently bad, as it is a necessary by-product in the break down of the essential amino acid methionine, which is found primarily in red meat and diary products. However, as with cholesterol, homocysteine may get out of balance as a result of genetics or poor diet. The main concern is having too much homocysteine. Low circulating levels of vitamin B6, B12 and folate have been associated with high homocysteine levels as well as elevated C-reactive protein levels. Hyperhomocysteinemia appears to enhance vascular inflammation and accelerates atherosclerosis. It has recently been discovered that folic acid, when combined with vitamins B6 and B12, has the potential of dramatically lowering the homocysteine levels, thereby protecting against high homocysteine-related diseases and occlusive vascular disease. Doses of folate at 500 to 5000 mcg daily and B12 at 500 mcg have been proposed as the necessary doses to lower homocysteine concentrations. Schynder and associates in 2002 reported the homocysteine lowering effects of folic acid (1000 mcg), vitamin B12 (400 mcg) and vitamin B6 (10 mg) in a randomized Heart Study investigating the clinical outcomes after percutaneous coronary intervention. (Schnyder G. “Effect of Homocysteine-Lowering therapy with folic acid, vitamin B12 and vitamin B6 on clinical outcomes after percutaneous coronary intervention. JAMA 2002; 288:973-97). The effect of this therapy at these doses appeared to significantly decrease the incidence of major adverse events after percutaneous coronary intervention. Friso et al. reported that low circulating levels of vitamin B6 have been associated with higher C-reactive levels independent of homocysteine levels, and B6 with these combination of B vitamins may be linked to an underlying inflammatory process when these levels are reduced. (S Friso et al. “Low circulating vitamin B6 is associated with elevation of the inflammation marker C-reactive protein independently of plasma homocysteine levels”. Circulation Vol 103; pp 2788-91 (2001)). The current DRI for folate is 180-200 mcg; for B6 or pyridoxine 1.6-2.0 mg and for vitamin B12 is 2.0-3.0 mcg or ug.

There exists a need for a nutritional supplement which supplies appropriate and effective amounts of calcium, vitamin D, and B vitamins for those who suffer from acne.

### SUMMARY OF THE INVENTION

This patent pertains to the micronutrient treatment of acne and inflammation. An object of this invention is to provide supplements, foodstuffs, beverages and beverage concentrates with calcium, vitamin D, folate, vitamin B12, vitamin B6 in the treatment and prevention of acne and inflammation.

Based upon observation of patients, it appears that the combination of adequate calcium, vitamin D and folate is essential in healthy skin and in the reduction of acne formation and inflammation. Relatively much lower doses (though higher than what is currently recommended by Al or RDA guidelines) of vitamin B12, vitamin B6 and riboflavin are protective but only in combination with fol ate at doses of 800 to 4000 mcg daily.

In accordance with the present invention, there is provided a multi-vitamin and mineral supplement which supplies appropriate and effective amounts of appropriate micronutrients at appropriate intervals to assure adequate intake of micronutrients needed for acne treatment and prevention against nutritional losses and deficiencies due to lifestyle factors and common inadequate dietary patterns.

Still further in accordance with the present invention, there is provided a multi-vitamin and mineral supplement wherein the supplement is comprised of 3 main ingredients calcium, vitamin D and folate: at 1000-1500 mg of elemental calcium in the form of carbonate or citrate or gluconate or citrate malate; and 400-4000 I.U. of parent vitamin D; and 800-4000 mcg of folic acid in combination with other B vitamins such as 25-100 mg of vitamin B6 (pyridoxine); 6-100 mcg of vitamin B12; and 50 mcg of riboflavin to satisfy normal daily metabolic balance requirements.

These and other advantages and benefits of the invention will be apparent to those skilled in the art upon reading and understanding of the following detailed description.

### DETAILED DESCRIPTION OF PREFERRED EMBODIMENTS

This invention is directed to a multi-vitamin and mineral supplement comprised of 1000-1500 mg of elemental calcium in the form of carbonate, citrate, gluconate or citrate malate; 400-4000 I.U. of vitamin D (parent vitamin D—cholecalciferol or ergocalciferol); 800-4000 mcg of folic acid (or folinic acid); 50 mg of vitamin B6; 30 mcg of vitamin B12; and 50 mcg of riboflavin. All amounts specified in the application are based on milligrams (mg); micrograms (mcg or ug) or International Units “I.U.”

The multi-vitamin and mineral supplement comprises vitamin D. Vitamin D is also an essential vitamin that is included in the multi-vitamin and mineral supplement of the present invention. Vitamin D assists in the mineralization and calcification of bone, prevents rickets in children, prevents osteomalacia in adults, preserves bone and tooth growth, and lowers blood pressure. Vitamin D is fat soluble. Preferably, the multi-vitamin and mineral supplement comprises about 400-4000 I.U. of vitamin D3 or cholecalciferol.

Vitamin D can reduce acneiform eruptions. An adequate amount of Vitamin D should be ingested in an amount to ensure a serum 25 vitamin D concentration between 30 ng-70 ng/ml. If the serum 25 hydroxyvitamin D concentration is above 30 ng/ml, the multi-vitamin and mineral supplement preferably is comprised of about 1000 I.U. of vitamin D in order to maintain optimal vitamin D concentrations. Preferably, in the multi-vitamin and mineral supplement, vitamin D is provided in the form of cholecalciferol at 1000 I.U. or 25 mcg (1 mcg=40 IU), to be taken on a daily basis. If the serum 25 hydroxyvitamin D concentration is below 30 ng/ml, the multi-vitamin and mineral supplement preferably is comprised of about 2000-4000 I.U. of vitamin D. Preferably, in the multi-vitamin and mineral supplement, vitamin D is provided in the form of cholecalciferol at 400-4000 I.U. (20-100 mcg) to be taken on a daily basis.

The vitamin D preferably is in the form of cholecalciferol (D3). As used herein, “vitamin D” comprises a group of, but not limited to, ergocalciferol (D2), cholecalciferol (D3), calcidiol (25 hydroxyvitamin D), or calcitriol (1,25 dihydroxyvitamin D).

Calcium, is the most important mineral in the body and is an important second messenger for numerous key cellular and enzyme functions, neuromuscular regulation and hormonal secretions, as well as for adequate bone health. It is the major bone mineral in the skeleton and is essential for optimal bone mineral acquisition. As used herein, calcium comprises elemental calcium in different forms, such as, but not limited to, calcium carbonate, calcium citrate, calcium citrate malate, calcium gluconate, calcium lactate, calcium acetate, or calcium stearate. The total daily micronutrient supplement is comprised of about 500 to 1500 elemental calcium daily.

The micronutrient/mineral supplement comprises some of the B complex of vitamins. The B vitamins are water-soluble. The B vitamins included in the mineral supplement are pyridoxine (vitamin B6), folic acid, the cobalamins (vitamin B12) and may even include riboflavin.

Folic acid or folate (also known as vitamin B9) is essential in the production of red blood cells, the production of hormones, and the synthesis of DNA. Preferably, the micronutrient/mineral supplement is comprised of about 800-4000 mcg of folic acid, but folinic acid, the metabolically more active form, may be used instead. The ratio of folate (mcg) to B12 (mcg) should be greater than 20 to 1.

Vitamin B6 or pyridoxine is involved in the production of ribonucleic acid (RNA) and deoxyribonucleic acid (DNA) and many other reactions in the body. Pyridoxine refers to and includes three different compounds: pyridoxine, pyridoxamine, and pyridoxal. Preferably, the vitamin B6 is in the form of pyridoxine hydrochloride. Preferably, the mineral supplement is comprised of about 25-100 mg of vitamin B6. More preferably, the micronutrient/mineral supplement is in the form of pyridoxine hydrochloride.

Vitamin B12 or the cobalamins are necessary for overall metabolism, the function of the nervous system, metabolism of folic acid, and the production of red blood cells. There are at least three active forms of cobalamin: cyanocobalamin, hydroxocobalamin, and nitrocobalamin. Preferably, in the multi-vitamin and mineral supplement of the present invention, vitamin B12 is provided in the form of cyanocobalamin. Preferably, the multi-vitamin and mineral supplement is comprised of about 20 mcg of vitamin B12. More preferably, the micronutrient/mineral supplement is comprised of about 6-50 mcg of vitamin B12 in the form of cyanocobalamin.

The nutritional supplements of the present invention are suitably provided in any suitable dosage form known in the art. For example, the compositions are suitably incorporated into tablets, powders, granules, beads, chewable lozenges, capsules, liquids, or similar conventional dosage forms, using conventional equipment and techniques known in the art. Tablet dosage forms are preferred.

When preparing dosages forms incorporating the compositions of the present invention, the nutritional components are normally blended with conventional excipients such as binders, including gelatin, pregelatinzed starch, and the like; lubricants, such as hydrogenated vegetable oil, stearic acid and the like; diluents, such as lactose, mannose, and sucrose; disintegants, such as carboxymethyl cellulose and sodium starch glycolate; suspending agents, such as povidone, polyvinyl alcohol, and the like; absorbents, such as silicon dioxide; preservative, such as methylparaben, propylparaben, and sodium benzoate; surfactants, such as sodium lauryl sulfate, polysorbate 80, and the like; and colorants, such as F.D & C. dyes and the like.

For preparing the composition from the compounds described by this invention, inert, pharmaceutically acceptable carriers are used which are either solid or liquid form. Solid form preparations include powders, tablets, dispersible granules, capsules, and cachets. A solid carrier is suitably one or more substances which may also act as diluents, flavoring agents, solubilizers, lubricants, suspending agents, binders or tablet disintegrating agents. The solid carrier material also includes encapsulating material. In powders, the carrier is finely divided active compounds. In the tablet, the active compound is mixed with the carrier having the necessary binding properties in suitable proportions and compacted into the shape and size desired. Suitable solid carriers include, but are not limited to, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term preparation is intended to include the formulation of the active compounds with encapsulating material as the carrier, providing a capsule in which the active component (with or without other carriers) is surrounded by carrier, which is thus in association with it. Tablets, powders, cachets, and capsules may be used in a solid dosage form suitable for oral administration.

Liquid form preparations include solutions, suspensions, and emulsions. Aqueous solutions suitable for oral use are prepared by dissolving the active component in water or other suitable liquid and adding suitable colorants, flavors, stabilizing agents, and thickening agents as desired. Aqueous solutions suitable for oral use may also be made by dispersing the finely divided active component in water or other suitable liquid with viscous material, such as natural or synthetic gums, resins, methylcellulose, sodium carboxymethylcellulose, and other suspending agents known in the art.

Also included are solid form preparations which are intended to be converted, shortly before use, to liquid form preparations for either oral or parental administration. Such liquid forms include solutions, suspensions, and emulsions. These particular solid form preparations are provided in unit dose form and as such are used to provide a single liquid dosage unit. Alternatively, sufficient solid preparation may be provided so that the after conversion to liquid form, multiple individual liquid doses may be obtained by measuring predetermined volumes of the liquid form preparation as with a syringe, teaspoon, or other volumetric contained.

The solid and liquid forms may contain, in addition to the active material, flavorants, colorants, stabilizers, buffers, artificial and natural sweeteners, dispersants, thickeners, solubilizing agents, and the like. The liquid utilized for preparing the liquid form preparation is suitably water, isotonic water, ethanol, glycerin, propylene glycol, and the like as well as combinations thereof. The liquid utilized will be chosen with regard to the route of administration.

Preferably, the preparations are unit dosage form. In such form, the preparation is subdivided into unit doses containing appropriate quantities of the active components. The unit dosage form can be a packaged preparation, such as packaged tablets or capsules. The unit dosage can be a capsule, cachet, or tablet itself or it can be the appropriate number of any of these in packaged form.

The quantity of active material in a unit dose of preparation is varied according to the particular application and potency of the active ingredients. Determination of the proper dosage for a particular situation is part of the present invention. For convenience, the total daily dosage may be divided and administered in portions during the day if desired. Controlled and uncontrolled release formulations are also included. When administered in combination, the amounts of calcium, vitamin D, vitamin B6 and fol ate thereof, may vary depending on serum concentration of some of these micronutrients and the mode of administration. For example, in one embodiment, the vitamin D is in an amount of about 2000 IU daily and the calcium is in the amount of 500 mg daily. If used in combination with vitamin D 1000, calcium 500 mg the total daily dose would be vitamin D 3000 IU, calcium 1000 mg.

Although the products of the invention are preferably intended for administration to humans, it will be understood that the formulation may also be utilized in veterinary therapy for other animals.

The present invention is further described in the following examples. It is understood that the example are only for illustrative purposes. The claims set forth the scope of the present invention.

### EXAMPLES

The effect of the micronutrient/mineral supplement of the present invention on acneiform eruptions is illustrated below.

1. AA was an 18 year old Indian female with a long history of cystic acne vulgaris. She had no history of polycystic ovarian syndrome, ovarian cysts, oligomenorrhea or amenorrhea. Topical benzoyl peroxide and antibiotics provided no relief of her acne. She refused both oral contraceptive use or a trial of the diuretic spironolactone for possible acne benefits. She was contemplating a trial of accutane, when she decided to see an endocrinologist for a possible hormone etiology of her skin condition. Complete blood count, chemistries, thyroid function tests, AM cortisol were normal. Vitamin B12 and folate concentrations were normal; the total testosterone was 40 ng and not elevated; the androgenic steroids—DHEAS and androstenedione were normal. The 25 hydroxyvitamin D concentration was <5 ng/ml and intact parathyroid hormone (PTH) was elevated at 69 pg/ml (normal up to 65 pg). She was prescribed calcium carbonate (elemental calcium 1500 mg), vitamin D3—cholecalciferol 4000 IU daily in combination with folate 1600 mcg, vitamin B6 50 mcg daily and vitamin B12 mcg 50 daily. The acneiform eruptions were markedly improved within 2 months. At four months, the 25 vitamin D concentration was 31 ng/ml and the total vitamin D3 was reduced to 2000 IU daily.

2. BB was a 30 year old Asian female with chronic fatigue, inability to lose weight and severe chronic acne vulgaris unresponsive to topical treatments. She had been on Accutane multilple times but her skin improvement was transient with continual periodic acne eruptions. Laboratory evaluation revealed normal thyroid function tests; total testosterone normal at 53 ng/dL; androstenedione normal at 234 ng/dL; DHEAS normal at 248 ug/dL; 17 hydroxyprogesterone normal at 92 ng/dL; normal AM cortisol; B12 low at −200 ng/L and normal serum folate level; 25 hydroxyvitamin D was low at 9 ng/ml (normal 10-68 ng) and intact parathyroid hormone level (PTH) was elevated at 105 pg/ml (normal up to 65 pg). She was prescribed vitamin D3 at 4000 IU daily with calcium carbonate (elemental calcium) at 1500 mg; folate 2000 IU; vitamin B6 at 50 mcg and vitamin B12 at 500 mcg daily (B12 was prescribed at 500 mcg for 2 weeks only because of B12 deficiency) and then lowered to 50 mcg daily). A repeat 25 vitamin D level at 3 months was 30 ng and the total daily vitamin D was then lowered to 2000 IU daily; repeat B12 level was normal. The acne eruptions were markedly reduced in 3 months.

3. CC was a 25 year old female with a known history of oligomenorrhea/amenorrhea since the age of 15. Her menstrual cycles had been irregular with fewer than 8 cycles per year. Oral contraceptives prescribed 5 years ago normalized her menstrual cycles. Over the past 2 years she complained of acne vulgaris despite different oral contraceptives and she had discontinued the oral contraceptives 4 months ago, while maintaining normal monthly menstrual cycles. She had initially been prescribed Accutane without success and was currently on the antibiotic minocycline without any effect on her acne. Her dietary calcium intake was very limited. Laboratory evaluation revealed normal thyroid function tests, total testosterone was normal at 32 ng/dL with free testosterone at 3.4 pg/ml; androstenedione was normal at 107 ng/dL; 17 hydroxyprogesterone was normal at 18 ng/dL; prolactin was 11 ug/L; B12 was 591 ng/L; serum folate >20 ng/ml; 25 vitamin D concentration was low normal at 30 ng/ml with intact PTH at 75.7 pg (normal up to 65 pg). Calcium carbonate (elemental calcium 1000 mg), vitamin D3-cholecalciferol 1000 IU; folate 1600 mcg with vitamin B6-pyridoxine 50 mcg resolved her acne in 2 months.

4. DD was a 16 year old male with a known history of metabolic bone disease and multiple skeletal fractures. His metabolic bone disease and osteoporosis were treated with cyclical courses of the bisphosphonate, Pamidronate (Aredia). His acne was severe and cystic involving his face, back and chest wall. He was placed on Minocin three times daily with topical micro retin A nightly with little improvement in his acne condition. Laboratory evaluation revealed normal complete blood count, sedimentation rate 7; normal chemistries and thyroid function tests; normal AM cortisol and normal cortisol following a cosyntropin stimulation test; total testosterone was low at 264 ng/dl and later rose to 318 ng; 25 vitamin was 21 ng/ml with slightly elevated intact PTH for his age at 60.1 pg; B12 was 822 ng/L; cortisol 13. 8; immunofixation was negative; serum folate was 484 ug/L. He was prescribed calcium carbonate (elemental calcium) 1000 mg, vitamin D3 initially at 2000 IU with folate 2000 mcg and vitamin B6 at 50 mcg with marked improvement of his acne. In 4 months, his 25 vitamin D level was 32 ng and the dose of vitamin D3 was reduced to 1000 IU.

5. EE was a 25 year old male who worked as a lifeguard during the summer months along the beaches of Coney Island in Brooklyn, N.Y. He complained of mild facial and back acne eruptions during most of the year, with moderate to severe eruptions particularly during the summer months when he was intensely exposed to the sun and also when he frequented tanning salons. He claimed his diet was excellent in adequate protein, fruits and vegetables with a daily milk intake of at least 3 glasses daily (equivalent to 900 mg of elemental calcium). He was baffled why even a cyclical course of antibiotics was unable to control his acne break outs. He sought an endocrine evaluation. Laboratory evaluation revealed normal complete blood count, normal chemistries, normal thyroid function tests, normal AM cortisol and normal cortisol following a cosyntropin stimulation test, normal testosterone at 620 ng/dl; normal 25 hydroxyvitamin D at 65 ng/ml and normal intact PTH at 32 pg/ml; normal serum folate >20 ng/ml; normal vitamin B12 at 730 ng/L; 24 hour urine calcium excretion was normal at 120 mg. He was prescribed elemental calcium 500 mg, folate 2000 mcg and pyridoxine 50 mg daily with marked improvement in his acne eruptions.

6. FF was a 19 year old male complaining of severe cystic acne involving his face and upper back. He had no history of major medical problems as diabetes mellitus, hypertension or colitis. Cyclical trials of both oral and topical antibiotics temporarily treated his skin condition but often resulted in frequent recurrences of his acne. His mother had coronary artery disease and was status post coronary angioplasty. She was told by her cardiologist to take the combination of the 3 B vitamins—folate at 1000 mcg, vitamin B12 at 500 mcg and pyridoxine at 50 mg once daily to lower her homocysteine levels and possibly reduce her risk of coronary events. Because of the effect on inflammation, she began her son on this same regimen which he inappropriately ingested 3-6 times daily for possibly faster acne benefits. Within two weeks, he suffered a very severe case of acne inflammation and eruptions. He was referred for both dermatologic and endocrine evaluations. Laboratory evaluation revealed normal complete blood count, chemistries, ESR 3; normal homocysteine level; normal thyroid function tests; normal folate >20 ng with vitamin B12 at >2000 ng/ml; normal AM cortisol and normal cortisol following a cosyntropin stimulation test; the 25 hydroxyvitamin D was low at 15 ng/ml and the intact parathyroid hormone was elevated at 108 pg/ml (normal up to 65 pg). He was told to immediately discontinue the high vitamin B complex regimen. Elemental calcium at 1500 mg and vitamin D3-4000 IU were prescribed and continued daily for 2 months. His inflammatory acne improved on this regimen, but not completely. Folate was restarted at 2000 mcg with vitamin B6—at 50 mcg; no vitamin B12 was prescribed. The vitamin D3 was reduced to 2000 IU when his 25 vitamin D concentration was >35 ng/ml. The daily high vitamin B complex ingestion with an abnormal ratio of folate to vitamin B12 to vitamin B6, probably resulted in the acute exacerbation of this patient's acne flare-up. Discontinuation of the high dose vitamin B12 and B6 combination with subsequent reinitiation of adequate folate, calcium and vitamin D supplementation markedly improved his acne.

The skin is the major site of the synthesis of vitamin D precursors and may even impact, through photodegradation, the catabolism of micronutrients such as folate. The combination of adequate calcium, vitamin D and folate appears essential in healthy skin and in the reduction of acne formation and inflammation. Relatively much lower doses (though higher than what is currently recommended by Al or RDA guidelines) of vitamin B12, vitamin B6 and riboflavin are protective but only in combination with folate at doses of 800 to 4000 mcg daily.

While various embodiments of a multi-vitamin and mineral supplement have been disclosed, it should be understood that modifications and adaptations thereof will occur to one skilled in the art. Other features and aspects of this invention will be appreciated by those skilled in the art upon reading and comprehending this disclosure. Such features, aspects, and expected variations and modifications of the reported results and examples are clearly within the scope of the invention where the invention is limited solely by the scope of the following claims.

1. A multi-vitamin and mineral supplement for administration to humans, the supplement comprising a combination effective for the treatment of acne comprising calcium, vitamin D, and folate.

2. The multi-vitamin and mineral supplement of claim 1 wherein the folate is folic acid or folinic acid.

3. The multi-vitamin and mineral supplement of claim 1 wherein the calcium is in the form selected from the group consisting of calcium carbonate, calcium citrate, calcium citrate malate, calcium gluconate, calcium lactate, calcium acetate, and calcium stearate, and wherein the total daily supplement is about 500 to 1500 mg of elemental calcium.

4. The multi-vitamin and mineral supplement of claim 1, suitable for administering a daily dosage wherein the calcium is 1000-1500 mg of elemental calcium in the form of a carbonate, citrate, gluconate or citrate malate, the vitamin D is 400-4000 I.U. of parent vitamin D, and the folate is 800-4000 mcg of folic acid.

5. The multi-vitamin and mineral supplement of claim 1, suitable for administering a daily dosage wherein the calcium is 1000-1500 mg of elemental calcium in the form of a carbonate, citrate, gluconate or citrate malate, the vitamin D is 400-4000 I.U. of vitamin D3 or cholecalciferol, the folate is 800-4000 mcg of folic acid.

6. The multi-vitamin and mineral supplement of claim 4, further comprising B vitamins to satisfy normal daily metabolic balance requirements.

7. The multi-vitamin and mineral supplement of claim 6, wherein the B vitamins are selected from the group consisting of vitamin B6 (pyridoxine), vitamin B12, and riboflavin.

8. The multi-vitamin and mineral supplement of claim 7 wherein the vitamin B6 is in the form of pyridoxine hydrochloride.

9. The multi-vitamin and mineral supplement of claim 8 wherein the vitamin B6 is 25-100 mg on a daily basis.

10. The multi-vitamin and mineral supplement of claim 8 wherein the vitamin B12 is in the form of cyanocobalamin between 6-100 mcg on a daily basis.

11. The multi-vitamin and mineral supplement of claim 10, wherein the cyanocabalamin or B12 is 6-20 mcg on a daily basis.

12. The multi-vitamin and mineral supplement of claim 7, wherein the ratio of folate (mcg) to vitamin B12 (mcg) is greater than 20 to 1.

13. The multi-vitamin and mineral supplement of claim 7, wherein the vitamin B6 is 25-100 mg, the vitamin B12 is 6-50 mcg, and the riboflavin is 50 mcg.

14. A for administration to humans, the supplement comprising a combination effective for the treatment of acne comprising calcium, vitamin D, and folate, wherein quantity of vitamin D assures a serum 25 hydroxyvitamin D concentration between 30 ng to 70 ng/ml.

15. The multi-vitamin and mineral supplement of claim 14 wherein for a 25 hydroxyvitamin D concentration below 30 ng the supplement comprises between 2000-4000 I.U. of vitamin D on a daily basis.

16. The multi-vitamin and mineral supplement of claim 14, wherein the vitamin D is in the form of cholecalciferol.

17. The multi-vitamin and mineral supplement of claim 15, wherein the vitamin D is in the form of cholecalciferol.

18. A method of supplementing the diet of humans at treating acne to achieve mineral and vitamin supplementation through the combination of the supplements contained in claim 1 whereby the resulting 25 hydroxyvitamin D level is achieved at, between, about 30-60 ng/ml.
